(19) United States
(12) Patent Application Publication
(10) Pub. No.: US 2019/0209502 A1
(43) Pub. Date: Jul. 11, 2019

(54) COMBINATION GEL FOR SEXUALLY TRANSMITTED INFECTIONS

(71) Applicant: EVOFEM, INC., San Diego, CA (US)

(72) Inventor: David R. Friend, San Diego, CA (US)

(21) Appl. No.: 16/332,290

(22) PCT Filed: Sep. 11, 2017
(86) PCT No.: PCT/US17/50960

§ 371 Date: Mar. 11, 2019

Related U.S. Application Data
(60) Provisional application No. 62/393,578, filed on Sep. 12, 2016.

(51) Int. Cl. A61K 31/19; A61K 47/12; A61K 47/14; A61K 47/10; A61K 47/36; A61P 31/20; A61P 31/18

(52) U.S. Cl. CPC A61K 31/19; A61K 47/12; A61K 47/14; A61K 9/0019; A61K 47/36; A61P 31/20; A61P 31/18; A61K 47/10

(57) ABSTRACT

Described herein are compositions comprising a combination of L-lactic acid and carrageenan and methods for treating or preventing HPV or other sexually transmitted infections by administering the composition.

### CROSS-REFERENCE

This application claims the benefit of U.S. Provisional Application No. 62/393,578 filed Sep. 12, 2016, which is incorporated by reference herein in its entirety.

### BACKGROUND OF THE DISCLOSURE

There is a need for developing compositions and methods for improved treatment and prevention of HPV and other sexually transmitted infections.

### SUMMARY OF THE DISCLOSURE

A composition for treating or preventing a sexually transmitted infection (STI) or a human papilloma virus infection (HPV), the composition comprising: (a) L-lactic acid and (b) carrageenan. In some embodiments, the composition further comprises: (c) a preservative, and (d) a polymer thickener. In some embodiments, the composition comprises (a) about 1-10% of the L-lactic acid, (b) about 0.1 to about 10% of the carrageenan, (c) about 0.1%-10% of the preservative, and (d) about 0.1%-10% of the polymer thickener. In some embodiments, the preservative is selected from a group consisting of benzoic acid, sodium benzoate, methylparaben, ethylparaben, butyulparaben, propylparaben, benzyalkonium chloride, phenylmercuric nitate, and chlorhexidine. In some embodiments, the polymer thickener is selected from a group consisting of xanthan gum, alginic acid, hydroxypropyl cellulose, hydroxypropyl methyl cellulose, sodium carboxymethyl cellulose, chitosan, polycarbophil, and carbopol. In some embodiments, the polymer thickener is a combination of xanthan gum and alginic acid. In some embodiments, the polymer thickener is alginic acid. In some embodiments, the carrageenan is a lambda carrageenan, kappa carrageenan, or an iota carrageenan. In some embodiments, the composition further comprising a humectant. In some embodiments, the humectant is selected from a group consisting of glycerol, polyethylene glycol, propylene glycol, sorbitol, and tiracetin. In some embodiments, the composition further comprises a pharmaceutically acceptable carrier. In some embodiments, the pharmaceutically acceptable carrier is an aqueous based carrier. In some embodiments, the composition further comprises one or more of water and a buffer, wherein the buffer comprises citric acid and potassium bitartrate. In some embodiments, the composition is a gel or a liquid. In some embodiments, the composition in injectable or instillable. In some embodiments, the composition is suitable for eradication of genital warts caused by HPV. In some embodiments, the STI comprises one or more of HIV, Hepatitis, Chancroid, Trichomoniasis, Herpes, Gonorrhea, Chlamydia, Bacterial Vaginosis, Syphilis, Scabies, Pelvic Inflammatory Disease, Mucopurulent Cervicitis, Molluscum Cotagiosum, and Lymphogranuloma Venereum.

One embodiment provides a method of treating or preventing a sexually transmitted infection (STI) or a human papilloma virus infection (HPV) comprising intravaginally administering a composition comprising: (a) L-lactic acid, and (b) carrageenan. In some embodiments, the composition further comprises one or more of: a preservative, a polymer thickener, a humectant, a pharmaceutically acceptable carrier, water, and a buffer. In some embodiments, the composition comprises (a) about 1-10% of the L-lactic acid, (b) about 0.1-10% of the carrageenan, (c) about 0.1-10% of the preservative, and (d) about 0.1-10% of the polymer thickener. In some embodiments, the composition is administered multiple times during a course of treatment. In some embodiments, the course of treatment comprises an initial course of treatment and a subsequent course of treatment, and wherein the composition is administered more frequently during the initial course of treatment than during the subsequent course of treatment. In some embodiment, the course of treatment is from about 1 week to about 20 weeks. In some embodiments, the frequency of administration during the initial course of treatment is once daily, less than once daily, or every other day. In some embodiments, the frequency of administration during the subsequent course of treatment is once a week or once every two weeks. In some embodiments, the composition is administered at a dosage from about 0.5 g to about 10 g, from about 3 g to about 5 g, about 3 g, about 4 g, or about 5 g. In some embodiments, the composition is administered by instillation or by injection. In some embodiments, administering the composition results in eradication of genital warts caused by HPV. In some embodiments, the STI comprises one or more of HIV, Hepatitis, Chancroid, Trichomoniasis, Herpes, Gonorrhea, Chlamydia, Bacterial Vaginosis, Syphilis, Scabies, Pelvic Inflammatory Disease, Mucopurulent Cervicitis, Molluscum Cotagiosum, and Lymphogranuloma Venereum. In some embodiments, the composition is administered within about 10 hours, within about 8 hours, without about 6 hours, or within about 2 hours subsequent to coitus.

Provided herein in one embodiment is a composition for treating or preventing a sexually transmitted infection (STI), the composition comprising: (a) L-lactic acid and (b) carrageenan. In some embodiments, the composition comprises: (a) L-lactic acid and (b) carrageenan. In some embodiments, the composition further comprises: (c) a preservative, and (d) a polymer thickener. In some embodiments, the composition comprises (a) about 1-10% of the L-lactic acid, (b) about 0.1 to about 10% of the carrageenan, (c) about 0.1% to about 10% of the preservative, and (d) about 0.1% to about 10% of the polymer thickener. In some embodiments, the preservative is selected from a group consisting of benzoic acid, sodium benzoate, methylparaben, ethylparaben, butyulparaben, propylparaben, benzyalkonium chloride, phenylmercuric nitrate, and chlorhexidine. In some embodiments, the polymer thickener is selected from a group consisting of xanthan gum, alginic acid, hydroxypropyl cellulose, hydroxypropyl methyl cellulose, sodium carboxymethyl cellulose, chitosan, polycarbophil, and carbopol. In some embodiments, the polymer thickener is a combination of xanthan gum and alginic acid. In some embodiments, the polymer thickener is alginic acid. In some embodiments, the alginic acid is Protacid F120. In some embodiments, the carrageenan is a lambda carrageenan, kappa carrageenan, or an iota carrageenan. In some embodiments, the composition further comprises a humectant. In some embodiments, the humectant is selected from a group consisting of glycerol, polyethylene glycol, propylene glycol, sorbitol, and tiracetin. In some embodiments, the composition further comprises a pharmaceutically acceptable carrier. In some embodiments, the pharmaceutically acceptable carrier is an aqueous based carrier. In some embodiments, the composition further comprises water. In some embodiments, the composition further comprises a buffer. In some embodiments, the buffer comprises citric acid and potassium bitartrate. In some embodiments, the composition is a gel. In some embodiments, the composition is a liquid. In some embodiments, the composition is injectable. In some embodiments, the composition is instillable. In some embodiments, the composition is suitable for eradication of genital warts caused by HPV. In some embodiments, the STI comprises HIV, Hepatitis, Chancroid, Trichomoniasis, Herpes, Gonorrhea, Chlamydia, Bacterial Vaginosis, Syphilis, Scabies, Pelvic Inflammatory Disease, Mucopurulent Cervicitis, Molluscum Cotagiosum, and Lymphogranuloma Venereum.

Provided herein in another embodiment is a method of treating or preventing a sexually transmitted infection (STI) comprising intravaginally administering a composition comprising: (a) L-lactic acid, and (b) carrageenan. In some embodiments, the method comprises, intravaginally administering a composition comprising: (a) L-lactic acid, and (b) carrageenan. In some embodiments, the composition further comprises: (c) a preservative, and (d) a polymer thickener. In some embodiments, the composition comprises (a) about 1-10% of L-lactic acid, (b) about 0.1 to about 10% of carrageenan, (c) about 0.1% to about 10% preservative, and (d) about 0.1% to about 10% polymer thickener. In some embodiments, the preservative is selected from a group consisting of benzoic acid, sodium benzoate, methylparaben, ethylparaben, butyulparaben, propylparaben, benzyalkonium chloride, phenylmercuric nitate, and chlorhexidine. In some embodiments, the polymer thickener is selected from a group consisting of xanthan gum, alginic acid, hydroxypropyl cellulose, hydroxypropyl methyl cellulose, sodium carboxymethyl cellulose, chitosan, polycarbophil, and carbopol. In some embodiments, the polymer thickener is a combination of xanthan gum and alginic acid. In some embodiments, the polymer thickener is alginic acid. In some embodiments, the alginic acid is Protacid F120. In some embodiments, the carrageenan is a lambda carrageenan, kappa carrageenan, or an iota carrageenan. In some embodiments, the composition further comprises a humectant. In some embodiments, the humectant is selected from a group consisting of glycerol, polyethylene glycol, propylene glycol, sorbitol, and tiracetin. In some embodiments, the composition further comprises a pharmaceutically acceptable carrier. In some embodiments, the pharmaceutically acceptable carrier is an aqueous based carrier. In some embodiments, the composition further comprises water. In some embodiments, the composition further comprises a buffer. In some embodiments, the buffer comprises citric acid and potassium bitartrate. In some embodiments, the composition is a gel. In some embodiments, the composition is a liquid. In some embodiments, the composition in injectable. In some embodiments, the composition is instillable. In some embodiments, the composition is administered multiple times during a course of treatment. In some embodiments, the course of treatment comprises an initial course of treatment and a subsequent course of treatment and wherein the composition is administered more frequently during the initial course of treatment than during the subsequent course of treatment. In some embodiments, the course of treatment is between 1 week and 20 weeks. In some embodiments, the frequency of administration during the initial course of treatment is once daily. In some embodiments, the frequency of administration during the initial course of treatment is every other day. In some embodiments, the frequency of administration during the subsequent course of treatment is once a week. In some embodiments, the frequency of administration during the subsequent course of treatment is once every two weeks. In some embodiments, the composition is administered at a dosage between about 0.5 g and about 10 g. In some embodiments, the composition is administered at a dosage between about 3 g and about 5 g. In some embodiments, the composition is administered at a dosage selected from about 3 g, about 4 g, and about 5 g. In some embodiments, the composition is administered by instillation. In some embodiments, the composition results in eradication of genital warts caused by HPV. In some embodiments, the STI comprises one or more of HIV, Hepatitis, Chancroid, Trichomoniasis, Herpes, Gonorrhea, Chlamydia, Bacterial Vaginosis, Syphilis, Scabies, Pelvic Inflammatory Disease, Mucopurulent Cervicitis, Molluscum Cotagiosum, and Lymphogranuloma Venereum.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF DRAWINGS

FIG. 1 illustrates effect of combination gel (CG) comprising L-lactic acid and carrageenan in decreasing HPV-16 pseudovirus infection in vivo. FIG. 1A shows mice treated with HEC, gel without carrageenan, or CG. FIG. 1B illustrates in vivo imaging results.

### DETAILED DESCRIPTION OF THE DISCLOSURE

While preferred embodiments of the present disclosure have been shown and described herein, it will be obvious to those skilled in the art that such embodiments are provided by way of example only. Numerous variations, changes, and substitutions will now occur to those skilled in the art without departing from the present disclosure. It should be understood that various alternatives to the embodiments of the present disclosure described herein may be employed. It is intended that the following claims define the scope of the present disclosure and that methods and structures within the scope of these claims and their equivalents be covered thereby.

Unless defined otherwise, all technical and scientific terms used herein have the same meaning as commonly understood by one of skill in the art to which the present disclosure described herein belong. All publications, patents, and patent applications mentioned in this specification are hereby incorporated by reference to the same extent as if each individual publication, patent, or patent application was specifically and individually indicated to be incorporated by reference.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose. The terminology used herein is for the purpose of describing particular cases only and is not intended to be limiting. As used herein, the singular forms “a”, “an” and “the” include the plural forms as well, unless the context clearly indicates otherwise. Furthermore, to the extent that the terms “contains,” “containing,” “including”, “includes,” “having,” “has”, “with”, or variants thereof are used in either the detailed description and/or the claims, such terms are intended to be inclusive in a manner similar to the term “comprising.”

The term “about” or “approximately” are meant to refer to values within an acceptable error range for the particular value as determined by one of ordinary skill in the art, which will depend in part on how the value is measured or determined, e.g., the limitations of the measurement system. For example, “about” means, in some cases, within 1 or more than 1 standard deviation, per the practice in the given value. Where particular values are described in the application and claims, unless otherwise stated the term “about” should be assumed to mean an acceptable error range for the particular value, such as ±10% of the value modified by the term “about”.

The terms “treat,” “treating,” and “treatment” include alleviating or abrogating a disorder, disease, or condition; or one or more of the symptoms associated with the disorder, disease, or condition; or alleviating or eradicating the cause(s) of the disorder, disease, or condition itself. Desirable effects of treatment include, but are not limited to, preventing occurrence or recurrence of disease, alleviation of symptoms, diminishing any direct or indirect pathological consequences of the disease, preventing metastasis, decreasing the rate of disease progression, amelioration or palliation of the disease state and remission or improved prognosis. The terms “prevent,” and “preventing,” include reducing the likelihood of occurrence of a disease, disorder, or condition.

Carrageenan, which is also known as Carregeenan, Chondrus extract, or Irish moss extract, is a hydrocolloid polysaccharide comprised primarily of potassium, sodium, calcium, magnesium, or ammonium sulfate esters of D-galactose and 3,6-anhydro-D-galactose copolymers. The pyranose moieties are linked by alternating alpha (1-->3) and beta (1-->4) glycosidic bonds. There exist at least three types of carrageenan, which are lambda-carrageenan, iota-carrageenan, and kappa-carrageenan, which differ in the amounts of sulfate ester and 3,6-anhydrogalactopyranose moieties. Lambda-carrageenan is a non-gelling polymer which contains about 35% sulfate ester groups by weight and no 3,6-anhydrogalactose moieties; iota-carrageenan is a gelling polymer which contains about 32% sulfate ester groups by weight and about 30% 3,6-anhydrogalactose moieties; and kappa-carrageenan is a comparatively stronger (i.e., inelastic, brittle or firm) gelling polymer which contains about 25% sulfate ester moieties by weight and about 34% 3,6-anhydrogalactose moieties. Carrageenan is available in a number of grades based on gelling type, aqueous solubility, and viscosity when blended with water and can be obtained from FMC Corporation under the trade names GELCARIN™, VISCARIN™, and SEASPEN™.

### Detailed Description of Composition

In some embodiments, the composition comprises about 0.1% to about 10% carrageenan and about 1% to about 10% L-lactic acid. In some embodiments, the composition comprises about 0.2% to about 9% carrageenan and about 1% to about 10% L-lactic acid. In some embodiments, the composition comprises about 0.3% to about 8% carrageenan and about 1% to about 10% L-lactic acid. In some embodiments, the composition comprises about 0.4% to about 7% carrageenan and about 1% to about 10% L-lactic acid. In some embodiments, the composition comprises about 0.5% to about 6% carrageenan and about 1% to about 10% L-lactic acid. In some embodiments, the composition comprises about 0.7% to about 5% carrageenan and about 1% to about 10% L-lactic acid. In some embodiments, the composition comprises about 0.8% to about 4% carrageenan and about 1% to about 10% L-lactic acid. In some embodiments, the composition comprises about 0.9% to about 4% carrageenan and about 1% to about 10% L-lactic acid. In some embodiments, the composition comprises about 1% to about 3% carrageenan and about 1% to about 10% L-lactic acid.

In some embodiments, the composition comprises about 0.1% to about 10% carrageenan and about 1% to about 9% L-lactic acid. In some embodiments, the composition comprises about 0.2% to about 9% carrageenan and about 1% to about 8% L-lactic acid. In some embodiments, the composition comprises about 0.3% to about 8% carrageenan and about 1% to about 7% L-lactic acid. In some embodiments, the composition comprises about 0.4% to about 7% carrageenan and about 1% to about 6% L-lactic acid. In some embodiments, the composition comprises about 0.5% to about 6% carrageenan and about 1% to about 5% L-lactic acid. In some embodiments, the composition comprises about 0.7% to about 5% carrageenan and about 1% to about 4% L-lactic acid. In some embodiments, the composition comprises about 0.8% to about 4% carrageenan and about 1% to about 3% L-lactic acid. In some embodiments, the composition comprises about 0.9% to about 4% carrageenan and about 1% to about 2% L-lactic acid. In some embodiments, the composition comprises about 1% to about 3% carrageenan and about 1% to about 1.5% L-lactic acid.

In some embodiments, the composition comprises about 0.1% to about 10% carrageenan and (a) about 1% to about 10% L-lactic acid, (b) about 0.1% to about 10% carrageenan, (c) about 0.1% to about 10% preservative, (d) about 0.1% to about 10% polymer thickener. In certain embodiments, the composition comprises 0.1% to about 10% carrageenan and (a) about 0.1% to about 10% carrageenan and (a) about 1% to about 10% L-lactic acid, (b) about 0.1% to about 5% carrageenan, (c) about 0.1% to about 2% preservative, (d) about 1% to about 7% polymer thickener. In certain embodiments, the composition comprises 0.1% to about 10% carrageenan and (a) about 1% to about 5% L-lactic acid, (b) about 0.5% to about 1.5% carrageenan, (c) about 0.1% to about 0.5% preservative, (d) about 1.5% to about 8% polymer thickener. In certain embodiments, the composition comprises 0.1% to about 10% carrageenan and (a) about 1% to about 2.5% L-lactic acid, (b) about 1% to about 2% carrageenan, (c) about 0.2% to about 0.4% preservative, (d) about 2% to about 5% polymer thickener. In certain embodiments, the composition comprises 0.1% to about 10% carrageenan and (a) about 1% to about 2% L-lactic acid, (b) about 0.5% to about 1% carrageenan, (c) about 0.1% to about 0.2% preservative, (d) about 2.5% to about 4.5% polymer thickener. In certain embodiments, the composition comprises 0.1% to about 10% carrageenan and (a) about 1% L-lactic acid, (b) about 1% carrageenan, (c) about 0.2% preservative, (d) about about 6.25% polymer thickener. In certain embodiments, the composition comprises 0.1% to about 10% carrageenan and (a) about 1% L-lactic acid, (b) about 1% carrageenan, (c) about 0.2% preservative, (d) about about 5.25% polymer thickener. The percent unit of the components refers to % weight/weight (% w/w).

Non-limiting examples of polymer thickeners include alginic acid, chitosan, gellan gum, poloxamer, hydroxypropyl cellulose, hydroxypropyl methyl cellulose, sodium carboxymethyl cellulose, polycarbophil, carbopol and the like. In some embodiments, the polymer thickener is alginic acid and xanthan gum. In some embodiments, the polymer thickener is alginic acid. Alginic acid is a generally linear glycouronan polymer containing a mixture of -(1,4)-D-gulosyuronic acid and -(1,4)-D-gulosyuronic acid residues. The molecular weight of the alginic acid is, in some instances, in the range of about 20,000 to about 300,000 g/mole, about 20,000 to about 250,000 g/mole, about 240,000 g/mole. about 25,000 to about 30,000 g/mole, about 30,000 to about 35,000 g/mole, about 35,000 to about 40,000 g/mole, about 40,000 to about 45,000 g/mole, about 45,000 to about 50,000 g/mole, about 50,000 to about 55,000 g/mole, about 55,000 to about 60,000 g/mole, about 60,000 to about 65,000 g/mole, about 65,000 to about 70,000 g/mole, about 70,000 to about 75,000 g/mole, about 75,000 to about 80,000 g/mole, about 80,000 to about 85,000 g/mole, about 85,000 to about 90,000 g/mole, about 95,000 to about 100,000 g/mole, about 125,000 to about 150,000 g/mole, about 150,000 to about 200,000 g/mole, or about 100,000 to about 200,000 g/mole. The average molecular weight of the alginic acid is, in some instances, in the range of about 20,000 to about 300,000 Da, about 20,000 to about 250,000 Da, about 240,000 Da, about 25,000 to about 30,000 Da, about 30,000 to about 35,000 Da, about 35,000 to about 40,000 Da, about 40,000 to about 45,000 Da, about 45,000 to about 50,000 Da, about 50,000 to about 55,000 Da, about 55,000 to about 60,000 Da, about 60,000 to about 65,000 Da, about 65,000 to about 70,000 Da, about 70,000 to about 75,000 Da, about 75,000 to about 80,000 Da, about 80,000 to about 85,000 Da, about 85,000 to about 90,000 Da, about 95,000 to about 100,000 Da, about 125,000 to about 150,000 Da, about 150,000 to about 200,000 Da, or about 100,000 to about 200,000 Da, about 180,000 Da, about 183,855 Da, about 185,000 Da, about 190,000 Da, or about 195,000 Da. In certain examples, the alginic acid comprises guluronic acid (G) and mannuronic acid (M) residues or units. In some instances, the alginic acid comprises about 65-75% G residues and about 25-35% M residues. The ratio between the M and G residues, are in some examples, from about 0.1 to 0.2, about 0.2 to about 0.3, about 0.3 to about 0.4, about 0.4 to about 0.5, about 0.5 to about 0.6, about 0.6 to about 0.7, about 0.7 to about 0.8, about 0.8 to about 1, about 0.2, about 0.21, about 0.22, about 0.23, about 0.24, about 0.25, about 0.26, about 0.27, about 0.28, about 0.29, about 0.30, about 0.31, about 0.32, about 0.33, about 0.34, about, 0.35, about 0.36, about 0.37, about 0.39, about 0.39, about 0.40, about 0.41, about 0.42, about 0.43, about 0.44, about 0.45, about 0.46, about 0.47, about 0.48, about 0.49, or about 0.5.

Alginic acid forms insoluble alginates by interacting with monovalent and divalent cations (especially Na⁺, K⁺, and Ca⁺⁺) in seminal plasma. Alginates also swell in contact with water, thereby assisting in maintaining the gel form of the composition within the vagina. It is also contemplated that alginic acid or salts of alginic acid contribute to the acid buffering activity of the composition since they have a pH of about 1.5 to about 3.5 in an aqueous solution. Furthermore, in some embodiments the alginic acid acts as a bioadhesive and provides the composition with bioadhesive properties. Without being bound by any particular theory, it is believed that because of its high molecular weight, alginic acid is not absorbed by the body. Thus, its effect as a polymer thickener, a bioadhesive, and acid-buffering agent is maintained so as long as the gel remains in the vagina. In some embodiments, the polymer thickener is xanthan gum. In some embodiments, the polymer thickener is a combination of xanthan gum and alginic acid. In some embodiments, the alginic acid is PROTACID F120.

The composition, in some embodiments, further comprises a bioadhesive compound. Non-limiting examples of bioadhesive compounds include, xanthan gum, hydroxypropyl cellulose, hydroxypropyl methyl cellulose, sodium carboxymethyl cellulose, chitosan, polycarbophil, carbopol, and the like. In some embodiments, the bioadhesive compound is xanthan gum, a high molecular weight polysaccharide gum containing D-glucosyl, D-mannosyl, and D-glucosyluronic acid residues and varying proportions of O-acetyl and pyruvic acid acetal. The primary structure of xanthan gum is a cellulose backbone with trisaccharide side chains; the repeating unit is a pentasaccharide. Generally, the molecular weight is greater than about 106 g/mole. In some embodiments, L-lactic acid is a buffering agent that acts to maintain the pH of the vagina within its normal acidic range (i.e., a pH of less than about 5 and more preferably in the range of about 3.5 to about 4.5). In some embodiments, the composition further comprises a buffer. Non-limiting examples of buffering agents include, but are not limited to, citric acid, potassium acid tartrate, benzoic acid, alginic acid, sorbic acid, fumaric acid, ascorbic acid, stearic acid, oleic acid, tartaric acid, edetic acid ethylenediaminetetracetic acid, acetic acid, malic acid, and the like. The acids may be added as free acids, hydrates, or pharmaceutically acceptable salts. It is understood that the free acids can be converted to the corresponding salts in situ (i.e., within the vagina). In some embodiments, several buffering agents are included in the combination gel to provide increased buffering capacity. In some embodiments, alginic acid, functions as both a polymer thickener and a buffering agent. Since alginic acid is absorbed by the body, its acid buffering effect is expected to be longer lasting as compared to the other buffering agents which may be absorbed by the body.

Additional excipients that may be used in the compositions of the present disclosure include humectants. Suitable humectants include, but are not limited to, for example, glycerol, polyethylene glycols, propylene glycols, sorbitol, triacetin, and the like. In some embodiments, glycerol is used as a humectant to prevent the formation of a dry film on the gel when placed within the vagina. In certain embodiments, glycerol also acts as a lubricant. Additionally, the combination gel, in some embodiments, also includes a preservative. Suitable preservatives include, but are not limited to, for example, benzoic acid, sodium benzoate, methylparaben, ethylparaben, butylparaben, propylparaben, benzyalkonium chloride, phenylmercuric nitrate, chlorhexidine, and the like. In some embodiments, the preservative is benzoic acid. In some embodiments, the combination gel comprises benzoic acid, which is a preservative and also contributes to the buffering capacity of the combination gel.

In some embodiments, the composition further comprises one or more cosmetic ingredients. Such cosmetic ingredients include diluents, solvents, and adjuvants, for example; water, ethyl alcohol, isopropyl alcohol, glycerin, glycerol propylene glycol, sorbitol, and other high molecular weight alcohols. In addition, the composition, in certain embodiments, further comprises minor amounts of other additives, such as, for example; stabilizers, surfactants, menthol, eucalyptus oil, other essential oils, fragrances, and the like. The selection and amounts of cosmetic ingredients, other additives, and blending procedures can be carried out in accordance with techniques well-known in the art.

In some embodiments, the pharmaceutical carrier is water. In some embodiments, the pharmaceutical carrier is aqueous based. Other pharmaceutically acceptable carriers that are suitable for vaginal delivery are well known and are used, in some examples, in place of water. One example of a suitable pharmaceutically acceptable carrier is petrolatum, such as white petrolatum.

In one exemplary embodiment of the present disclosure, the combination gel is further described as follows: the polymer thickener is alginic acid and xanthan gum; the preservative is benzoic acid; the humectant is glycerol; citric acid, potassium bitartrate, and lactic acid are acidic components; and water is the pharmaceutically acceptable carrier. In another embodiment, the composition contains xanthan gum, alginic acid, L-lactic acid, citric acid, benzoic acid, potassium bitartrate, glycerol, and water. In another embodiment, the lactic acid is L-lactic acid.

As discussed, lactic acid or other suitable buffering agents are used to maintain the pH of the vagina within its normal acidic range (i.e., a pH of less than about 5 and more preferably in the range of about 3.5 to about 4.5).

The present disclosure identifies that administering the combination gel comprising L-lactic acid and carrageenan surprisingly increases vaginal defense mechanisms against various sexually transmitted infections, including human papilloma virus (HPV). The combination of L-lactic acid and carrageenan results in greater inactivation of microbes, including viruses, in comparison to compounds such as hydrogen peroxide or acetic acid at equivalent pH.

Lactic acid has two isomers, one is known as L-(+)-lactic acid or (S)-lactic acid and the other is D-(−)-lactic acid or (R)-lactic acid. Recent discovery has shown that the L form of lactic acid is more potent in inactivating HIV than D or racemic lactic acid. While the precise mechanism of how L-lactic acid invactivates HIV is unknown, the stereo chemical dependent activity suggests that it acts on proteins.

The composition of the present disclosure is in the form of a gel, a semi-solid, a cream, a lotion, and/or a liquid. In some embodiments, the composition is injected. In some embodiments, the composition is instilled. In some embodiments, the composition is administered as a topical ointment applied to the lining of the vagina and/or cervix and/or rectum, which can be accomplished as a gel, cream, lotion, non-aqueous or aqueous solution used to flush the vaginal or rectal cavity, and/or a vaginal or rectal suppository. In other embodiments, the composition is administered in a spray formulation.

In some embodiments, the compositions of the present disclosure is delivered to the vagina of a mammal by any means known to those skilled in the art. Typical forms for delivery of the compositions include, for example; creams, lotions, gels, foams, intravaginal devices such as sponges and suppositories, and films. In addition, the composition can be used as personal care products, such as, for example, condom lubricants, and the like. Such lubricants may comprise commonly known ingredients such as, for example: humectants, e.g., glycerin, sorbitol, mannitol, glycols and glycol ethers; buffers, e.g., glucono-d-lactone; germicides or bactericides, e.g., chlorhexidine gluconate; preservatives, e.g., methylparaben; viscosifiers, e.g., hydroxyethyl cellulose, etc.; other adjuvants, e.g., colors and fragrances; in addition to the compositions of the present disclosure. Those skilled in the art will recognize that the physical properties, e.g., viscosity, of such delivery forms may vary widely. For example, the viscosity of a gel form of the composition of the present disclosure, e.g., 150,000 centipoise, may be substantially higher than the viscosity of lotion form of the composition of the present disclosure, e.g., 100 centipoise. Further details concerning the materials, ingredients, proportions and procedures of such delivery forms can be selected in accordance with techniques known in the field.

### Method of Treatment and Prevention

Provided herein in one embodiment is a method of treating or preventing a sexually transmitted infection (STI) comprising intravaginally administering a composition comprising: (a) L-lactic acid, and (b) carrageenan. In another embodiment, the disclosure comprises a method of treating or preventing a human papilloma virus infection (HPV) comprising intravaginally administering a composition comprising: (a) L-lactic acid, and (b) carrageenan. In some embodiments, the composition is a gel. In some embodiments, the composition is a liquid. In some embodiments, the composition is a gel that is administered by injection or instillation. In some embodiments, the composition is a liquid that is administered by injection or instillation. The injection or instillation is on the vaginal tissue.

In some embodiments, the methods described herein comprise administration of the disclosed compositions subsequent to coitus. In some embodiments, the composition is administered within about 10 hours subsequent to coitus. In some embodiments, the composition is administered within about 8 hours subsequent to coitus. In some embodiments, the composition is administered within about 6 hours subsequent to coitus. In some embodiments, the composition is administered within about 4 hours subsequent to coitus. In some embodiments, the composition is administered within about 2 hours subsequent to coitus.

The methods described herein comprise administration of the disclosed compositions at frequent intervals during a course of treatment. In some embodiments, the course of treatment comprises an initial course and a subsequent course and the composition is administered more frequently during the initial course compared to the subsequent course. In certain embodiments, the course of treatment is about 1 week to about 20 weeks. In certain embodiments, the course of treatment is about 2 weeks to about 19 weeks, about 3 weeks to about 18 weeks, about 4 weeks to about 17 weeks, about 5 weeks to about 16 weeks, about 6 weeks to about 15 weeks, about 7 weeks to about 14 weeks, about 8 weeks to about 13 weeks, about 9 weeks to about 12 weeks, about 10 weeks to about 11 weeks, about 1 week, about 2 weeks, about 3 weeks, about 4 weeks, about 5 weeks, about 6 weeks, about 7 weeks, about 8 weeks, about 9 weeks, about 10 weeks about 11 weeks, about 12 weeks, about 13 weeks, about 14 weeks, about 15 weeks, about 16 weeks, about 17 weeks, about 18 weeks, about 19 weeks, about 20 weeks, about 21 weeks, about 22 weeks, about 23 weeks, about 24 weeks, about 7 months, about 8 months, about 9 months, about 10 months, about 11 months, about 12 months, about 13 months, or from about 6 months to about 24 months. In certain embodiments, the course of treatment is about 3 weeks to about 16 weeks. In some cases, the initial course of treatment is about 1 week, about 2 weeks, about 3 weeks, about 4 weeks, about 5 weeks, about 6 weeks, about 7 weeks, about 8 weeks, about 9 weeks, about 10 weeks, about 11 weeks, or about 12 weeks. In certain examples, the subsequent course of treatment is about 1 week, about 2 weeks, about 3 weeks, about 4 weeks, about 5 weeks, about 6 weeks, about 7 weeks, about 8 weeks, about 9 weeks, about 10 weeks, about 11 weeks, or about 12 weeks, about 13 weeks, about 14 weeks, about 15 weeks, about 16 weeks, about 17 weeks, about 18 weeks, about 19 weeks, about 20 weeks, about 21 weeks, about 22 weeks, about 23 weeks, about 24 weeks, about 7 months, about 8 months, about 9 months, about 10 months, about 11 months, about 12 months, about 13 months, or from about 6 months to about 24 months.

In some embodiments, the frequency of administration during the initial course is daily, every other day, or once a week. In some cases, the subsequent course of treatment comprises about 1 week and the frequency of administration is less than once daily. In some embodiments, the frequency of administration during the initial course is once a day, once every 12 hours, once every 6 hours, once every 4 hours, or once every 2 hours. In some embodiments, the frequency of administration during the initial course is such that there is no more than 24 hours gap between administration of two doses. In some embodiments, the frequency of administration during the initial course is such that there is no more than 48 hours gap between administration of two doses. In some embodiments, the frequency of administration during the initial course is such that there is no more than 72 hours gap between administration of two doses. In some embodiments, the frequency of administration during the initial course is such that there is no more than 96 hours gap between administration of two doses. In some embodiments, the frequency of administration during the initial course is such that there is no more than 120 hours gap between administration of two doses. In some embodiments, the frequency of administration during the initial course is such that there is no more than 168 hours gap between administration of two doses. In other embodiments, the frequency of administration during the initial course is such that there is more than 24 hours gap between administration of two doses. In other embodiments, the frequency of administration during the initial course is such that there is more than 48 hours gap between administration of two doses. In other embodiments, the frequency of administration during the initial course is such that there is more than 72 hours gap between administration of two doses. In other embodiments, the frequency of administration during the initial course is such that there is more than 96 hours gap between administration of two doses. In other embodiments, the frequency of administration during the initial course is such that there is more than 120 hours gap between administration of two doses. In other embodiments, the frequency of administration during the initial course is such that there is more than 168 hours gap between administration of two doses. In some cases, the initial course of treatment comprises about 1 week and the frequency of administration is once daily. In some cases, the initial course of treatment comprises about 1 week and the frequency of administration is such that there is more than 24 hours gap between administration of two doses. In some cases, the initial course of treatment comprises about 1 week and the frequency of administration is such that there is more than 48 hours gap between administration of two doses. In some cases, the initial course of treatment comprises about 1 week and the frequency of administration is such that there is more than 72 hours gap between administration of two doses. In some cases, the initial course of treatment comprises about 1 week and the frequency of administration is such that there is more than 96 hours gap between administration of two doses. In some cases, the initial course of treatment comprises about 1 week and the frequency of administration is such that there is more than 120 hours gap between administration of two doses. In some cases, the initial course of treatment comprises about 1 week and the frequency of administration is such that there is no more than 168 hours gap between administration of two doses. In some cases, the initial course of treatment comprises about 1 week and the frequency of administration is such that one dose is administered during the week. In some examples, the initial course of treatment comprises about 2 weeks and the frequency of administration is such that one dose is administered during the 2 weeks. In some cases, the initial course of treatment comprises about 2 weeks and the frequency of administration is such that at least two doses are during the 2 weeks.

In some embodiments, the frequency of administration during the subsequent course is every other day, once a week, or twice a week. In some embodiments, the frequency of administration during the subsequent course is such that there is more than 24 hours gap between administration of two doses. In some embodiments, the frequency of administration during the subsequent course is such that there is more than 48 hours gap between administration of two doses. In some embodiments, the frequency of administration during the subsequent course is such that there is more than 72 hours gap between administration of two doses. In some embodiments, the frequency of administration during the subsequent course is such that there is more than 96 hours gap between administration of two doses. In some embodiments, the frequency of administration during the subsequent course is such that there is more than 120 hours gap between administration of two doses. In some embodiments, the frequency of administration during the subsequent course is such that there is more than 168 hours gap between administration of two doses. In some cases, the subsequent course of treatment comprises about 1 week and the frequency of administration is less than once daily. In some cases, the subsequent course of treatment comprises about 1 week and the frequency of administration is such that there is more than 24 hours gap between administration of two doses. In some cases, the subsequent course of treatment comprises about 1 week and the frequency of administration is such that there is more than 48 hours gap between administration of two doses. In some cases, the subsequent course of treatment comprises about 1 week and the frequency of administration is such that there is more than 72 hours gap between administration of two doses. In some cases, the subsequent course of treatment comprises about 1 week and the frequency of administration is such that there is more than 96 hours gap between administration of two doses. In some cases, the subsequent course of treatment comprises about 1 week and the frequency of administration is such that there is more than 120 hours gap between administration of two doses. In some cases, the subsequent course of treatment comprises about 1 week and the frequency of administration is such that there is no more than 168 hours gap between administration of two doses. In some cases, the subsequent course of treatment comprises about 1 week and the frequency of administration is such that one dose is administered during the week. In some examples, the subsequent course of treatment comprises about 2 weeks and the frequency of administration is such that one dose is administered during the 2 weeks. In some cases, the initial course of treatment comprises about 2 weeks and the frequency of administration is such that at least two doses are administered during the 2 weeks. In some cases, the initial course of treatment comprises about 3 weeks and the frequency of administration is such that only one dose is administered during the three weeks.

In some embodiments, the compositions of the present disclosure are administered to the vagina of the mammal in a dosage comprising about 0.5 g to about 10 g, about 2 g to about 9 g, about 3 g to about 8 g, about 4 g to about 7 g, about 5 g to about 6 g, about between 3 g to about 4 g, or about 4 g to about 5 g of the composition. The dosage of administration, in some embodiments comprises about 0.5 g, about 0.6 g, about 0.7 g, about 0.8 g, about 0.9 g, about 1 g, about 1.1 g, about 1.2 g, about 1.3 g, about 1.4 g, about 1.5 g, about 1.6 g, about 1.7 g, about 1.8 g, about 1.9 g, about 2 g, about 2.1 g, about 2.2 g, about 2.3 g, about 2.4 g, about 2.5 g, about 2.6 g, about 2.7 g, about 2.8 g, about 2.9 g, about 3 g, about 3.1 g, about 3.2 g, about 3.3 g, about 3.4 g, about 3.5, about 3.6 g, about 3.7 g, about 3.8, about 3.9, about 4 g, about 5 g, about 5.1 g, about 5.2 g, about 5.3 g, about 5.4 g, about 5.5 g, about 5.6 g, about 5.7 g, about 5.8 g, about 5.9 g, about 6 g, about 7 g, about 7.1 g, about 7.2 g, about 7.3 g, about 7.4 g, about 7.5 g, about 7.6 g, about 7.7 g, about 7.8, about 7.9 g, about 8 g, about 8.1 g, about 8.2 g, about 8.3 g, about 8.4 g, about 8.5 g, about 8.6 g, about 8.7 g, about 8.8 g, about 8.9 g, about 9 g, about 9.1 g, about 9.2 g, about 9.3, about 9.4 g, about 9.5 g, about 9.6 g, about 9.7, about 9.8 g, about 9.9 g, about 10 g, about 10.1 g, about 10.2 g, about 10.3 g, about 10.4 g, about 10.5 g, about 10.6 g, about 10.7 g, about 10.8 g, about 10.9 g, about 11 g, about 11.1 g, about 11.2 g, about 11.3 g, about 11.4 g, about 11.5 g, about 11.6 g, about 11.7 g, about 11.8 g, about 11.9 g, about 12 g, about 12.1 g, about 12.2 g, about 12.3 g, about 12.4 g, about 12.5 g, about 12.6 g, about 12.7 g, about 12.8 g, about 12.9 g, about 13 g, about 13.1 g, about 13.2 g, about 13.3 g, about 13.4 g, about 13.5 g, about 13.6 g, about 13.7 g, about 13.8 g, about 13.9 g, about 14 g, about 14.1 g, about 14.2 g, about 14.3 g, about 14.5 g, about 14.6 g, about 14.7 g, about 14.8 g, about 14.9 g, about 15 g, about 15.1 g, about 15.2 g, about 15.3 g, about 15.4 g, about 15.5 g, about 15.6 g, about 15.7 g, about 15.8 g, about 15.9 g, about 16 g, 16.1 g, about 16.2 g, about 16.3 g, about 16.4 g, about 16.5 g, about 16.6 g, about 16.7 g, about 16.8 g, about 16.9 g, about 17 g, about 17.1 g, about 17.2 g, about 17.3 g, about 17.4 g, about 17.5 g, about 17.6 g, about 17.7 g, about 17.8 g, about 17.9 g, about 18 g, about 18.1 g, about 18.2 g, about 18.3 g, about 18.4 g, about 18.5 g, about 18.6 g, about 18.7 g, about 18.8 g, about 18.9 g, about 19 g, about 19.1 g, about 19.2 g, about 19.3 g, about 19.4 g, about 19.5 g, about 19.6 g, about 19.7 g, about 19.8 g, about 19.9 g, or about 20 g of the composition. In certain cases, the composition is administered at a dosage comprising from about 10 μg to about 400 μg L-lactic acid, about 20 μg to about 200 μg L-lactic acid, about 30 μg to about 100 μg L-lactic acid, about 40 μg to about 50 μg L-lactic acid, about 60 μg L-lactic acid, about 70 μg L-lactic acid, about 75 μg L-lactic acid, about 80 μg L-lactic acid, about 85 μg L-lactic acid, about 90 μg L-lactic acid, or about 100 μg L-lactic acid. In certain cases, the composition is administered at a dosage comprising from about 5 μg to about 200 μg carrageenan, about 10 μg to about 150 μg carrageenan, about 15 μg to about 100 μg carrageenan, about 20 μg to about 75 μg carrageenan, about 30 μg carrageenan, about 40 μg carrageenan, about 50 μg carrageenan, about 60 μg carrageenan, about 65 μg carrageenan, about 75 μg carrageenan, about 100 μg carrageenan, or about 200 μg carrageenan.

In some embodiments, the composition is administered to treat or prevent HPV in a mammal. In some embodiments, administering the composition results in eradication of genital warts caused by HPV. In some embodiments, the composition is administered to treat or prevent a sexually transmitted infection (STI) in a mammal. In some embodiments, the STI comprises one or more of HIV, Hepatitis, Chancroid, Trichomoniasis, Herpes, Gonorrhea, Chlamydia, Bacterial Vaginosis, Syphilis, Scabies, Pelvic Inflammatory Disease, Mucopurulent Cervicitis, Molluscum Cotagiosum, and Lymphogranuloma Venereum.

### EXAMPLES

The following specific, non-limiting examples are to be construed as merely illustrative, and do not limit the present disclosure of the scope of the disclosure. Without further elaboration, it is believed that one skilled in the art can, based on the description herein, utilize the present disclosure to its fullest extent.

### Example 1: Method of Preparing a Composition Comprising L-Lactic Acid and Carrageenan

A 500 mL beaker is set up with an Ultra Turax and 25 mm HSM attachment. The following items were added sequentially: Water, USP; ION NaOH; Benzoic acid. The solution was transferred to a Camframo 2002 equipped with a side sweep mixing attachment. The next few ingredients (potassium bitartrate; citric acid, 88% L-lactic acid; alginic acid; carrageenan; glycerin). The pH was tested and adjusted to 3.5±0.2 with ION NaOH if out of range. Xanthan gum was added to the solution and subsequently the solution was mixed for at least 60 minutes.

Exemplary composition included the following:

| Ingredient | Concentration % w/w |
|---|---|
| Benzoic Acid | 0.20 |
| 10N NaOH | 2.20 |
| Purified Water USP | 74.00 |
| Potassium Bitartrate | 0.40 |
| Citric Acid | 1.00 |
| Lactic Acid 88% Solution | 2.00 |
| Alginic Acid (Protacid F120) | 4.25 |
| Carrageenan | 1.00 |
| Glycerin | 8.00 |
| Xanthan Gum | 2.00 |
| 10N NaOH for adjusting pH to 3.5 | As needed |

### Example 2: In Vivo Anti-HPV Activity of Combination Gel Comprising L-Lactic Acid and Carrageenan

### SUMMARY

Animal models for HPV involve the use of HPV pseudoviruses (HPV PsV), which contain a plasmid with reporter genes within the viral capsid composed of the L1 and L2 proteins. The HPV PsVs have the ability to mimic the early steps of infection from viral adsorption to entry into a basal keratinocyte where the reporter gene (such as luciferase) is expressed. Viral infection can then be determined by quantifying the reporter gene using in vivo imaging techniques. Additionally, seminal plasma can be used with the viral inoculum (HPV PsVs) to test any possible detrimental effects on the potential microbicide. The models are a good tool to test formulations that may block viral adsorption and/or entry but not any of the subsequent steps in the viral replication cycle.

Experimental Protocol

Virus

HPV-16 PsV was produced following the NCI protocol published on Dr. John Schiller's laboratory website (available at http://home.ccr.cancer.gov/Lco/). For this purpose, 293TT cells were obtained from NCI at Frederick, Md., and the following plasmids were generously provided by Dr. John Schiller or purchased through Addgene, Cambridge, Mass.: p16shell and Addgene plasmid 37328. Aliquots of virus stocks were stored at −80° C.

HPV-16 PsV Vaginal Challenge in Mice

Depo-Provera-treated Balb/C mice were treated intravaginally with Conceptrol 6 hours before HPV PsV challenge. The Conceptrol treatment promoted abrasions in the epithelium, allowing the binding of HPV PsV to the basement membrane. 20 μL of test formulation (gel without carrageenan, combination gel comprising L-Lactic acid and carrageenan, or hydroxycellulose (HEC) placebo) were inserted intravaginally 1 h before vaginal challenge with 8×10⁶ copies/10 μL of HPV-16 PsV. A group of 5 animals received 20 μL of Dulbecco's phosphate-buffered saline (D-PBS) intravaginally without HPV PsV challenge to use as a negative control in the imaging system. In vivo luciferase expression was measured 24 h after intravaginal challenge by anesthetizing the animals with isoflurane (Aerrane, Deerfield, Ill.), applying 20 μL of D-luciferin (Caliper Life Sciences, Hopkinton, Mass.) intravaginally, incubating 3 min and performing in vivo imaging in the IVIS® Spectrum imaging system (Perkin Elmer, Waltham, Mass.). Luminescence signal was expressed as mean value in radiance.

Statistical Analysis

The Mann Whitney U test was used for comparison of in vivo luciferase expression in the HPV PsV mouse model. Statistical analysis was performed using GraphPad Prism version 5.02 for Windows (GraphPad Software, San Diego, Calif.). P values <0.05 were taken as statistically significant.

Results

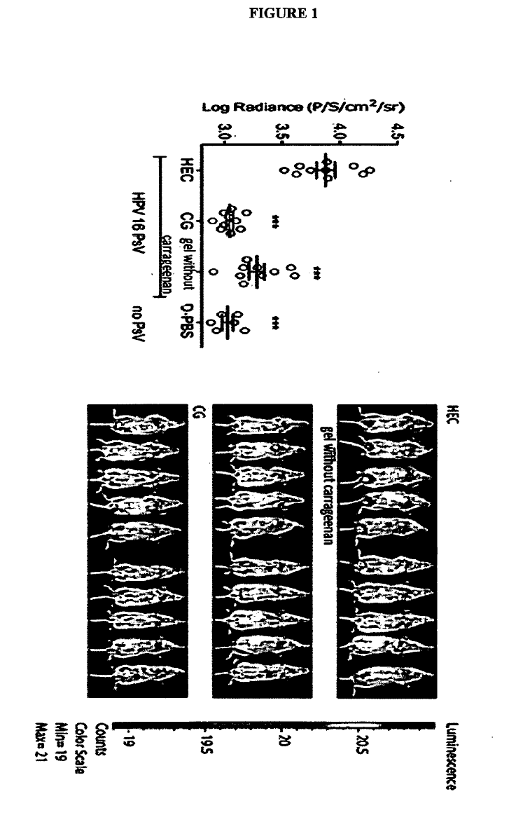

A significant decrease in HPV 16 PsV infection in the vaginal murine model was observed when the combination gel (CG) comprising L-lactic acid and carrageenan was applied vaginally 1 h before virus challenge (p<0.000). The results are illustrated in FIGS. 1A and 1B.

FIG. 1 shows that the combination gel (CG) significantly decreases HPV16 PsV infection in mice. A) Depo-Provera-treated Balb/C mice were given HEC, gel without carrageenan, or combination gel (CG) comprising L-lactic acid and carrageenan, intravaginally at 1 h before HPV 16 PsV challenge or D-PBS without HPV 16 PsV challenge (n=6-10/treatment). In vivo luciferase expression was detected using IVIS® Spectrum imaging system and is expressed as mean luminescence in photons per second per cm2 per steridian±SD for each individual animal. Statistical analysis was performed using the Mann-Whitney test (*** p<0.0001). B) Imaging results in the IVIS® Spectrum imaging system.

### CONCLUSION

The study suggested that the new combination gel comprising L-lactic acid and carrageenan administered one hour before HPV PsV challenge had significant in vivo antiviral activity against HPV, compared to a gel without the carrageenan and the placebo.

### CERTAIN EMBODIMENTS

Embodiment 1 provides a composition for treating or preventing a sexually transmitted infection (STI), the composition comprising: (a) L-lactic acid and (b) carrageenan.

Embodiment 2 provides a composition for treating or preventing a human papilloma virus infection (HPV), the composition comprising: (a) L-lactic acid and (b) carrageenan.

Embodiment 3 provides the composition of embodiment 1 or 2, further comprising: (c) a preservative, and (d) a polymer thickener.

Embodiment 4 provides the composition of any one of embodiments 1-3, comprising (a) about 1-10% of the L-lactic acid, (b) about 0.1 to about 10% of the carrageenan, (c) about 0.1% to about 10% of the preservative, and (d) about 0.1% to about 10% of the polymer thickener.

Embodiment 5 provides the composition of any one of embodiments 1-4, wherein the preservative is selected from a group consisting of benzoic acid, sodium benzoate, methylparaben, ethylparaben, butyulparaben, propylparaben, benzyalkonium chloride, phenylmercuric nitate, and chlorhexidine.

Embodiment 6 provides the composition of any one of embodiments 1-5, wherein the polymer thickener is selected from a group consisting of xanthan gum, alginic acid, hydroxypropyl cellulose, hydroxypropyl methyl cellulose, sodium carboxymethyl cellulose, chitosan, polycarbophil, and carbopol.

Embodiment 7 provides the composition of any one of embodiments 1-6, wherein the polymer thickener is a combination of xanthan gum and alginic acid.

Embodiment 8 provides the composition of any one of embodiments 1-7, wherein the polymer thickener is alginic acid.

Embodiment 9 provides the composition of any one of embodiments 1-8, wherein the alginic acid is Protacid F120.

Embodiment 10 provides the composition of any one of embodiments 1-9, wherein the carrageenan is a lambda carrageenan, kappa carrageenan, or an iota carrageenan.

Embodiment 11 provides the composition of any one of embodiments 1-10, wherein the carrageenan is the iota carrageenan.

Embodiment 12 provides the composition of any one of embodiments 1-11, further comprising a humectant.

Embodiment 13 provides the composition of any one of embodiments 1-12, wherein the humectant is selected from a group consisting of glycerol, polyethylene glycol, propylene glycol, sorbitol, and tiracetin.

Embodiment 14 provides the composition of any one of embodiments 1-13, further comprising a pharmaceutically acceptable carrier.

Embodiment 15 provides the composition of any one of embodiments 1-14, wherein the pharmaceutically acceptable carrier is an aqueous based carrier.

Embodiment 16 provides the composition of any one of embodiments 1-15, further comprising water.

Embodiment 17 provides the composition of any one of embodiments 1-16, further comprising a buffer.

Embodiment 18 provides the composition of any one of embodiments 1-17, wherein the buffer comprises citric acid and potassium bitartrate.

Embodiment 19 provides the composition of any one of embodiments 1-18, wherein the composition is a gel.

Embodiment 20 provides the composition of any one of embodiments 1-18, wherein the composition is a liquid.

Embodiment 21 provides the composition of any one of embodiments 1-20, wherein the composition in injectable.

Embodiment 22 provides the composition of any one of embodiments 1-20, wherein the composition is instillable.

Embodiment 23 provides the composition of any one of embodiments 1-22, wherein the composition is suitable for eradication of genital warts caused by HPV.

Embodiment 24 provides the composition of any one of embodiments 1, and 3-22, wherein STI comprises HIV, Hepatitis, Chancroid, Trichomoniasis, Herpes, Gonorrhea, Chlamydia, Bacterial Vaginosis, Syphilis, Scabies, Pelvic Inflammatory Disease, Mucopurulent Cervicitis, Molluscum Cotagiosum, or Lymphogranuloma Venereum.

Embodiment 25 provides a method of treating or preventing a sexually transmitted infection (STI) comprising intravaginally administering a composition comprising: (a) L-lactic acid, and (b) carrageenan.

Embodiment 26 provides a method of treating or preventing a human papilloma virus infection (HPV) comprising intravaginally administering a composition comprising: (a) L-lactic acid, and (b) carrageenan.

Embodiment 27 provides the method of any one of embodiments 25-26, wherein the composition further comprises: (c) a preservative, and (d) a polymer thickener.

Embodiment 28 provides the method of any one of embodiments 25-27, wherein the composition comprises (a) about 1-10% of the L-lactic acid, (b) about 0.1 to about 10% of the carrageenan, (c) about 0.1% to about 10% of the preservative, and (d) about 0.1% to about 10% of the polymer thickener.

Embodiment 29 provides the method of any one of embodiments 25-28, wherein the preservative is selected from a group consisting of benzoic acid, sodium benzoate, methylparaben, ethylparaben, butyulparaben, propylparaben, benzyalkonium chloride, phenylmercuric nitate, and chlorhexidine.

Embodiment 30 provides the method of any one of embodiments 25-29, wherein the polymer thickener is selected from a group consisting of xanthan gum, alginic acid, hydroxypropyl cellulose, hydroxypropyl methyl cellulose, sodium carboxymethyl cellulose, chitosan, polycarbophil, and carbopol.

Embodiment 31 provides the method of any one of embodiments 25-30, wherein the polymer thickener is a combination of xanthan gum and alginic acid.

Embodiment 32 provides the method of any one of embodiments 25-31, wherein the polymer thickener is alginic acid.

Embodiment 33 provides the method of any one of embodiments 25-32, wherein the alginic acid is Protacid F120.

Embodiment 34 provides the method of any one of embodiments 25-33, wherein the carrageenan is a lambda carrageenan, a kappa carrageenan, or an iota carrageenan.

Embodiment 35 provides the method of any one of embodiments 25-34, wherein the carrageenan is the iota carrageenan.

Embodiment 36 provides the method of any one of embodiments 25-35, wherein the composition further comprises a humectant.

Embodiment 37 provides the method of any one of embodiments 25-36, wherein the humectant is selected from a group consisting of glycerol, polyethylene glycol, propylene glycol, sorbitol, and tiracetin.

Embodiment 38 provides the method of any one of embodiments 25-37, wherein the composition further comprises a pharmaceutically acceptable carrier.

Embodiment 39 provides the method of any one of embodiments 25-38, wherein the pharmaceutically acceptable carrier is an aqueous based carrier.

Embodiment 40 provides the method of any one of embodiments 25-39, wherein the composition further comprises water.

Embodiment 41 provides the method of any one of embodiments 25-40, wherein the composition further comprises a buffer.

Embodiment 42 provides the method of any one of embodiments 25-41, wherein the buffer comprises citric acid and potassium bitartrate.

Embodiment 43 provides the method of any one of embodiments 25-42, wherein the composition is a gel.

Embodiment 44 provides the method of any one of embodiments 25-42, wherein the composition is a liquid.

Embodiment 45 provides the method of any one of embodiments 25-44, wherein the composition is injectable.

Embodiment 46 provides the composition of any one of embodiments 25-44, wherein the composition is instillable.

Embodiment 47 provides the method of any one of embodiments 25-46, wherein the composition is administered multiple times during a course of treatment.

Embodiment 48 provides the method of any one of embodiments 25-47, wherein the course of treatment comprises an initial course of treatment and a subsequent course of treatment, and wherein the composition is administered more frequently during the initial course of treatment than during the subsequent course of treatment.

Embodiment 49 provides the method of any one of embodiments 25-48, wherein the course of treatment is from about 1 week to about 20 weeks.

Embodiment 50 provides the method of any one of embodiments 25-49, wherein the frequency of administration during the initial course of treatment is once daily.

Embodiment 51 provides the method of any one of embodiments 25-50, wherein the frequency of administration during the initial course of treatment is every other day.

Embodiment 52 provides the method of any one of embodiments 25-51, wherein the frequency of administration during the subsequent course of treatment is once a week.

Embodiment 53 provides the method of any one of embodiments 25-52, wherein the frequency of administration during the subsequent course of treatment is once every two weeks.

Embodiment 54 provides the method of any one of embodiments 25-53, wherein the composition is administered at a dosage from about 0.5 g to about 10 g.

Embodiment 55 provides the method of any one of embodiments 25-54, wherein the composition is administered at a dosage from about 3 g to about 5 g.

Embodiment 56 provides the method of any one of embodiments 25-55, wherein the composition is administered at a dosage selected from about 3 g, about 4 g, and about 5 g.

Embodiment 57 provides the method of any one of embodiments 25-56, wherein the composition is administered by instillation.

Embodiment 58 provides the method of any one of embodiments 25-57, wherein administering the composition results in eradication of genital warts caused by HPV.

Embodiment 59 provides the method of any one of embodiments 25 and 27-58, wherein STI comprises one or more of HIV, Hepatitis, Chancroid, Trichomoniasis, Herpes, Gonorrhea, Chlamydia, Bacterial Vaginosis, Syphilis, Scabies, Pelvic Inflammatory Disease, Mucopurulent Cervicitis, Molluscum Cotagiosum, and Lymphogranuloma Venereum.

Embodiment 60 provides the method of any one of embodiments 25-59, wherein the composition is administered subsequent to coitus.

Embodiment 61 provides the method of embodiment 60, wherein the composition is administered within about 10 hours subsequent to coitus.

Embodiment 62 provides the method of embodiment 60, wherein the composition is administered within about 8 hours subsequent to coitus.

Embodiment 63 provides the method of embodiment 60, wherein the composition is administered within about 6 hours subsequent to coitus.

Embodiment 64 provides the method of embodiment 60, wherein the composition is administered within about 4 hours subsequent to coitus.

Embodiment 65 provides the method of embodiment 60, wherein the composition is administered within about 2 hours subsequent to coitus.

1. A composition for treating or preventing a sexually transmitted infection (STI) or a human papilloma virus infection (HPV), the composition comprising: (a) L-lactic acid and (b) carrageenan.

2. The composition of claim 1, further comprising: (c) a preservative, and (d) a polymer thickener.

3. The composition of claim 2, comprising (a) about 1-10% of the L-lactic acid, (b) about 0.1 to about 10% of the carrageenan, (c) about 0.1%-10% of the preservative, and (d) about 0.1%-10% of the polymer thickener.

4. The composition of claim 2, wherein the preservative is selected from a group consisting of benzoic acid, sodium benzoate, methylparaben, ethylparaben, butyulparaben, propylparaben, benzyalkonium chloride, phenylmercuric nitate, and chlorhexidine.

5. The composition of claim 2, wherein the polymer thickener is selected from a group consisting of xanthan gum, alginic acid, hydroxypropyl cellulose, hydroxypropyl methyl cellulose, sodium carboxymethyl cellulose, chitosan, polycarbophil, and carbopol.

6. The composition of claim 5, wherein the polymer thickener is a combination of xanthan gum and alginic acid.

7. The composition of claim 6, wherein the polymer thickener is alginic acid.

8. The composition of claim 1, wherein the carrageenan is a lambda carrageenan, kappa carrageenan, or an iota carrageenan.

9. The composition of claim 1, further comprising a humectant.

10. The composition of claim 9, wherein the humectant is selected from a group consisting of glycerol, polyethylene glycol, propylene glycol, sorbitol, and tiracetin.

11. The composition of claim 1, further comprising a pharmaceutically acceptable carrier.

12. The composition of claim 11, wherein the pharmaceutically acceptable carrier is an aqueous based carrier.

13. The composition claim 1, further comprising one or more of water and a buffer, wherein the buffer comprises citric acid and potassium bitartrate.

14. The composition of claim 1, wherein the composition is a gel or a liquid.

15. The composition of claim 1, wherein the composition in injectable or instillable.

16. The composition of claim 1, wherein the composition is suitable for eradication of genital warts caused by HPV.

17. The composition of claim 1, wherein STI comprises one or more of HIV, Hepatitis, Chancroid, Trichomoniasis, Herpes, Gonorrhea, Chlamydia, Bacterial Vaginosis, Syphilis, Scabies, Pelvic Inflammatory Disease, Mucopurulent Cervicitis, Molluscum Cotagiosum, and Lymphogranuloma Venereum.

18. A method of treating or preventing a sexually transmitted infection (STI) or a human papilloma virus infection (HPV) comprising intravaginally administering a composition comprising: (a) L-lactic acid, and (b) carrageenan.

19. (canceled)

20. (canceled)

21. The method of claim 18, wherein the composition is administered multiple times during a course of treatment.

22. The method of claim 21, wherein the course of treatment comprises an initial course of treatment and a subsequent course of treatment, and wherein the composition is administered more frequently during the initial course of treatment than during the subsequent course of treatment.

23-30. (canceled)
